(19) United States
(12) Patent Application Publication
(10) Pub. No.: US 2010/0146646 A1
(43) Pub. Date: Jun. 10, 2010

(54) METHOD OF MONITORING RETINOPATHY

(76) Inventors: Lang Zhuo, Singapore (SG); Gideon Ho, Singapore (SG); Saravana Kumar, Singapore (SG)

Correspondence Address: KLARQUIST SPARKMAN, LLP, 121 SW SALMON STREET, SUITE 1600, PORTLAND, OR, 97204, US

(21) Appl. No.: 12/598,593

(22) PCT Filed: May 2, 2008
(86) PCT No.: PCT/SG08/00159

§ 371 Date: Nov. 2, 2009

Related U.S. Application Data
(60) Provisional application No. 60/924,161, filed on May 2, 2007.

(51) Int. Cl. G01N 33/53

(52) U.S. Cl. 800/3

(57) ABSTRACT

There is presently provided methods of monitoring retinopathy in a live transgenic, non-human animal, the methods comprising providing a live transgenic non-human animal having a retinal pathology or a pre-disposition for a retinal pathology, wherein a nucleic acid molecule encoding a fluorescent protein under control of a GFAP promoter is integrated into the genome of the transgenic non-human animal; and detecting in vivo in the retinal glia of the transgenic non-human animal fluorescence levels of the fluorescent protein.

### CROSS-REFERENCE TO RELATED APPLICATION

This application claims benefit of, and priority from, U.S. provisional patent application No. 60/924,161, filed on May 2, 2007, the contents of which are incorporated herein by reference.

### FIELD OF THE INVENTION

The present invention relates to methods of monitoring retinopathy in the retina in vivo.

### BACKGROUND OF THE INVENTION

Non-invasive fluorescent molecular imaging of gliotic reaction in the retinas of experimental models of retinopathy is of great interest for in vivo pre-clinical screening for “primary” retinopathies (originating from eye disorders) and for “secondary” retinopathies (originating from systemic disorders in organs other than eye), as well as for monitoring the efficacy and possible toxicity of therapeutic candidates.

Various ophthalmic conditions as well as numerous systemic diseases outside the eye can cause retinopathy, which is a non-inflammatory degenerative disease of the retina that leads to visual field loss or blindness.

Many retinal disorders can be diagnosed with the aid of retinal and/or optic nerve examination. Examples of such disorders include hypertension, a chronic increase in systemic blood pressure associated with peripheral arteriole constriction (Tien Yin Wong, 2004; Hammond S, 2006; Topouzis F, 2006), vascular diseases (Yamakawa K, 2001; McCulley T J, 2005), congenital heart disease (Mansour et al., 2005), autoimmune diseases such as rheumatoid arthritis, which causes chronic inflammation of the joints and other body parts (Giordano N, 1990; Aristodemou P, 2006), multiple sclerosis which involves destruction of the myelin sheaths of neurons in the central nervous system (CNS) (Lucarelli M J, 1991; Kenison J B, 1994; Lycke J, 2001), neurofibromatosis (Chan C C, 2002; Karadimas P, 2003; Ruggieri M, 2004), Lyme neuroborreliosis (Burkhard et al., 2001), Down's syndrome (Satge D, 2005; Liza-Sharmini A T, 2006), autism (Ek et al., 1998), sickle cell anaemia (Sandstrom H, 1997; Chalam K V, 2004; Shakoor A, 2005; Lima C S, 2006), infections like HIV (A R Irvine, 1997; Kozak I, 2005; V T Pham1, 2005) and cytomegalovirus (Vogel J U, 2005; Zhang M, 2005a; Zhang M, 2005b), diabetes (Antonetti D A, 2006; Takahashi H, 2006; Vujosevic S, 2006), thyroid disorders (Bahceci U A, 2005; Mader M M, 2006; Roberts M R, 2006), liver disorders (Heidrun Kuhrt, 2004; Colakoglu Onder, 2005), eye diseases such as retinoschisis (Kirsch et al., 1996), age-related macular degeneration (Guidry et al., 2002) and glaucoma (Wang L, 2002) and neurodegenerative diseases (Blanks J C, 1996a; Helmlinger D, 2002).

Methods for in vivo imaging of the retina have commonly been reflection imaging or contrast-injected fluorescence angiography (Hawes N L, 1999; Bruce E. Cohan, 2003), but these methods do not allow visualization of changes at the molecular level. Furthermore, potential scarring, leakage and inflammatory reactions are incurred on injection of contrast agents, affecting the results obtained (Ritter et al., 2005).

Thus, there exists a need for a retinopathy model that allows for in vivo, non-invasive monitoring of retinopathy status, for applications relating to disease progression, prognosis, and assessment of potential treatment efficacy and toxicity.

### SUMMARY OF THE INVENTION

The present invention provides, in one aspect, a method for monitoring retinopathy, comprising: providing a live transgenic non-human animal having a retinal pathology or a pre-disposition for a retinal pathology, wherein a nucleic acid molecule encoding a fluorescent protein under control of a GFAP promoter is integrated into the genome of the transgenic non-human animal; and detecting in vivo in the retinal glia of the transgenic non-human animal a first fluorescence level of the fluorescent protein at a first time point and a second fluorescence level of the fluorescent protein at a second time point.

The present invention further provides, in another aspect, a method for monitoring retinopathy, comprising: providing a live first transgenic non-human animal having a retinal pathology or a pre-disposition for a retinal pathology, wherein a nucleic acid molecule encoding a fluorescent protein under control of a GFAP promoter is integrated into the genome of the first transgenic non-human animal; providing a live second transgenic non-human animal that is free from a retinal pathology or a pre-disposition for a retinal pathology, wherein a nucleic acid molecule encoding a fluorescent protein under control of a GFAP promoter is integrated into the genome of the second transgenic non-human animal; and detecting in vivo in the retinal glia of the first transgenic non-human animal a first fluorescence level of the fluorescent protein and in the retinal glia of the second transgenic non-human animal a second fluorescence level of the fluorescent protein.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention:

FIG. 1: Fluorescent molecular imaging of saline-treated and neurotoxicant KA induced GFP elevation in the optic disc of the adult mouse retina. GFP fluorescence reaches a maximum around the fringe of the optic disc at Day 7.

FIG. 2: Tiled images of whole-mount retina showing KA induced gliosis in the nerve fiber layer (NFL) of the retina 7 days after a single ip injection. Basal level of GFP and GFAP expression can be seen in the whole-mount retina of the saline-treated control mouse; bar=200 μm.

FIG. 3: Retinal vasculature in the gliotic retina of the KA-treated mouse (right merged image) enveloped by both the processes and cell bodies of the reactive astrocytes at 7 days after a single neurotoxicant ip injection (indicated by arrows). Astrocytic cell bodies and processes are labeled by transgenic GFP but GFAP labeling is typically confined to the processes (indicated by arrowheads). Basal level of GFP and GFAP (red) expression can be seen in the whole-mount retina of the saline-treated control mouse (left merged image); bar=50 μm.

FIG. 4: A series of confocal images focused at the optic disc of the KA-treated mouse at regularly placed intervals through the depth of the whole-mount retina show induced gliosis at 7 days following a single ip injection of the neurotoxicant. Extent of gliosis is shown in astrocytic cell bodies and processes from the nerve fiber layer (NFL) and ganglion cell layer at approximately 0-15 μm into the inner plexiform layer (15-20 μm). In addition, clearly shown at 20 μm is where the transverse view of reactive processes of Müller cells is prominent in area around the optic disc. Various depth information on transgenic GFP level of expression can also be seen in the whole-mount retina of the saline-treated control mouse; bar=100 μm.

FIG. 5: Neurotoxicant KA induced severe gliosis in the astrocytes (green, ˜10 μm in size) of the entire hippocampus 7 days after administration of the neurotoxicant as revealed by transgenic GFP fluorescence and GFAP antibody staining (red). Basal level of GFP and GFAP expression can be seen in the hippocampus of the saline-treated control mouse brain; bar=200 μm. Cells double-labeled by GFP and GFAP are indicated in yellow in the merged image.

FIG. 6: Neurotoxicant KA induced gliosis in the CA1 region of the hippocampus 7 days after administration of the neurotoxicant. Basal level of GFP and GFAP expression can be seen in the CA1 region of the saline-treated control mouse brain; bar=100 μm.

FIG. 7: Neurotoxicant KA induced gliosis in the CA3 region of the hippocampus 7 days after administration of the neurotoxicant. Base-line level of GFP and GFAP expression can be seen in the CA3 region of the saline-treated control mouse brain; bar=100 μm.

FIG. 8: Neurotoxicant KA induced gliosis in the dentate gyrus region of the hippocampus 7 days after administration of the neurotoxicant. Base-line level of GFP and GFAP expression can be seen in the dentate gyrus of the saline-treated control mouse brain; bar=100 μm.

FIG. 9: Fluorescent molecular imaging of saline-treated and neurotoxicant 2′-CH₃-MPTP induced GFP elevation in the optic disc of the adult mouse retina. GFP fluorescence reaches a maximum around the fringe of the optic disc at Day 1.

FIG. 10: Neurotoxicant 2′-CH₃-MPTP induced gliosis in the astrocytes (green, ˜10 μm in size) of the substantia nigra a day after administration of the neurotoxicant as revealed by transgenic GFP fluorescence and GFAP antibody staining (red). Basal level of GFP and GFAP expression can be seen in the substantia nigra of the saline-treated control mouse brain. Some cells double-labeled by GFP and GFAP are indicated by arrows; bar=50 μm.

FIG. 11: Neurotoxicant 2′-CH₃-MPTP induced gliosis in the astrocytes with an observable tyrosine hydroxylase (TH) depletion in the dopaminergic neurons (red, ˜20 μm in size) a day after administration of the neurotoxicant in the substantia nigra pars compacta (SNpc) area of the neurotoxicant-treated brain. A base-line control level of GFP and TH expression was detected in the SNpc of the saline-treated mouse brain; bar=50 μm.

FIG. 12: In the striatum of the neurotoxicant-treated brain, similar to the neurotoxicity effects of 2′-CH₃-MPTP in the midbrain SNpc, acute gliosis was observed a day after administration of the neurotoxicant in the transgenic GFP striatal astrocytes. Due to the low basal expression level of TH in the striatum, no change in TH expression could be visually observed in accompanying the acute gliosis in the GFP-labeled astrocytes; bar=50 μm.

FIG. 13: Fluorescent molecular imaging of saline-treated and neurotoxicant IDPN induced GFP elevation in the optic disc of the adult mouse retina. GFP fluorescence reaches a maximum around the fringe of the optic disc at Day 7.

FIG. 14: Neurotoxicant IDPN induced acute gliosis mainly in the astrocytes (green, ˜10 μm in size) of the glomerular layer (GL) of the olfactory bulb (indicated by arrows in the merged images) at 7 days following the administration of the neurotoxicant as revealed by transgenic GFP fluorescence and GFAP antibody staining (red). A base-line control level of GFP and GFAP expression can be seen in the olfactory bulb of the saline-treated mouse brain; bar=100 μm.

FIG. 15: Neurotoxicant IDPN induced gliosis in the astrocytes of the GL and granular cell layer (GCL) of the olfactory bulb 7 days after administration of the neurotoxicant. Basal level of GFP and GFAP expression can be seen in the olfactory bulb of the saline-treated control mouse brain. Cells double-labeled by GFP and GFAP are indicated in yellow in the merged image; bar=200 μm.

### DETAILED DESCRIPTION

The present invention relates to models of retinopathy that allow for monitoring of the disease in vivo, in a non-invasive manner, including at the molecular level. Such in vivo models provide methods to study disease progression and treatment of diseases and disorders stemming from causes such as neurodegenerative diseases and neurotoxicity. Methods are described herein for imaging the retinal glia in an animal model, and such methods may provide real-time utility leading to diagnosis of primary and secondary retinopathies, as well as for the evaluation of efficacy and neurotoxicity of therapeutic compounds. These methods are non-therapeutic methods designed to assist in characterising and understanding retinopathy, even though in some instances potential therapies or treatments may be tested using the in vivo non-human animal model, for example for the purposes of assessing the efficacy, effect or toxicity of any potential therapy or treatment.

The retina is composed of several layers of neurons (including the ganglion cell layer (GCL)) and glia (including astrocytes and Müller cells). The ganglion cells transmit the signal from the preceding photoreceptor cells via axons to the optic nerve, then to the brain. Since the retina and optic nerve are embryonic outgrowths of the brain, they have often been used as a simple model for the CNS. The clear optical media of the eye allows for direct visualization of labeled disease processes as they develop, making the retina and the optic nerve the most accessible components for in vivo study of the CNS. Since the retina is continuous with the CNS, it is also afflicted in neurodegenerative diseases such as Alzheimer and Parkinson's, and thus represents the status of the CNS. In the case of the Alzheimer's, it has been shown that there is an overall 25% decrease in retinal ganglion cells compared with age matched controls (Blanks J C, 1996b).

Since the retina is a highly organized and homogeneous tissue with a limited number of different cell types, monitoring of the retina in retinopathy related diseases may facilitate the identification of cells involved in the particular disease pathology, in contrast to other regions of the CNS (Morgan J., 2005). An assessment of the retina is therefore a useful tool for determining the extent of an underlying disease in a non-invasive manner and may aid determination of prognosis and monitoring of disease progression in a patient. Due to its accessibility, the retina not only allows local application of therapeutic vectors (gene transfer and drug delivery) with reduced risk of systemic effects, but also facilitates the assessment of therapeutic strategies and medical trials (Helmlinger D, 2002).

The inventors have developed a method to monitor retinopathy status using imaging of a fluorescent reporter protein, such as green fluorescent protein (GFP), coupled to the glial fibrillary acidic protein (GFAP) promoter in an in vivo animal model of retinopathy. GFAP is expressed in degenerative retinopathy and thus serves as a specific biomarker linked to retinopathy disease status. The present in vivo methods allow for greater morphological detail and progressive pathological assessment of retinopathy and potential treatments over the course of the disease, and thus are useful to assess and characterise different disease stages, including onset, progression, regression and recovery. The present methods are performed in vivo in a live animal, and thus may be performed over a period of time in the same animal, over which time period the disease status may change.

GFAP is found predominantly in normal and reactive glial cells of the CNS, which responds to injury during gliosis by up-regulating GFAP. In the retina, astrocytes and Müller cells are the glial cell type (Dyer M A, 2000), and normally contain low levels of GFAP. However, in response to stress or injury, these cells demonstrate substantial increases in GFAP production, leading to cellular proliferation and change in shape (Milena Kuzmanovic, 2003). Previous studies have shown that GFAP can be imaged on normal glial cells and those undergoing degeneration in fixed tissues or live tissues ex vivo (Brenner M, 1994; Blanks J C, 1996a; Cordeiro et al., 2004; Morgan J., 2005). Here, the inventors have demonstrated that a fluorescent marker protein expressed under control of the GFAP promoter can be used to image the retina in vivo in a live animal having retinal degeneration or retinopathy or a pre-disposition to develop retinal degeneration or retinopathy, including over a period of time in order to monitor retinopathy disease status.

For monitoring the status or progress of neurodegeneration, the present methods utilize a live small animal disease model expressing a fluorescent protein, such as GFP, under GFAP promoter control. That is, a GFAP-GFP transgenic animal having retinal pathology, induced for example by genetic or chemical techniques, or a pre-disposition to develop a retinal pathology, provides a live animal model for in vivo retinal imaging to monitor retinopathy or retinopathy related diseases or disorders, or to assess efficacy or toxicity of treatment in retinopathy or retinopathy related diseases or disorders.

An advantage of these methods over the histological method (Seeliger et al., 2005) lies in the simplicity of tracking a pathological progression in vivo over time, in a system that can respond to treatment or which can show further degeneration, in real-time and without the need for an exogenous dye.

Thus, there is provided a method of monitoring retinopathy, comprising detecting fluorescence levels of a fluorescent protein in the glia of the retina (i.e. Müller cells and astrocytes) and including the optic nerve (non-myelinating Schwann cells) of a transgenic non-human animal having a retinal pathology or a pre-disposition for a retinal pathology including retinal degeneration and which transgenic animal expresses a fluorescent protein under control of the GFAP promoter.

The expression of the fluorescent protein is “under control” of the GFAP promoter in the transgenic animal, meaning that the GFAP promoter is operably linked to the coding sequence for the fluorescent protein and is the promoter that directs transcription of the fluorescent protein coding sequence. Thus, factors that activate or enhance transcription from the GFAP promoter may result in increased expression of the fluorescent protein in the transgenic animal, and factors that inhibit or block transcription from the GFAP promoter may result in decreased expression of the fluorescent protein in the transgenic animal, relative to expression in the absence of any such factors. Expression levels of the fluorescent protein are detected non-invasively by detecting fluorescence levels of the fluorescent protein in the retinal glia of the transgenic animal.

In order to monitor retinopathy over a desired time period, a first fluorescence level of the fluorescent protein may be detected at a first time point, and then a second fluorescence level of the fluorescent protein may be detected at a second time point, in the same live animal. The second fluorescence level may be compared to the first fluorescence level in order to assess a change in disease status.

As well, monitoring of retinopathy may be done in comparison to a negative control animal model, in order to assess disease status. Thus, a transgenic non-human animal expressing the fluorescent protein under control of the GFAP promoter, but which does not have a retinal pathology or a pre-disposition for a retinal pathology, may be used to provide a standard of protein fluorescence in the retina that is not related to retinopathy, thus allowing for comparison of disease status in an animal having or being pre-disposed for a retinal pathology and an animal being free from any such pathology or pre-disposition.

Monitoring retinopathy includes tracking disease onset, progression, regression, recovery or prognosis over a period of time and also includes tracking of response to treatment and tracking of side effects, including toxicity, of treatment, over a period of time.

The retinopathy may be any retinopathy, including primary retinopathy or secondary retinopathy, and may be the result of a disease or genetic condition in the transgenic animal or may be chemically- or radiation-induced by treating the transgenic animal with a neurotoxin or radiation, as described below.

Thus, monitoring retinopathy may include monitoring and quantifying the fluorescence levels of fluorescent protein in order to assess disease status of the retina in any retinopathy related disease or disorder.

A retinopathy related disease or disorder refers to any disease, disorder or condition which may cause, result in, or is associated with retinal degeneration, retinal gliosis or retinopathy, including a primary retinopathy or a secondary retinopathy.

A retinal pathology includes damage, degeneration or disease of the retina that is the result of or is related to a retinopathy or a retinopathy related disease or disorder, and including degeneration of the retinal glia. Reference to a pre-disposition for a retinal pathology refers to an increased risk for, a susceptibility to, or a tendency (including a genetic tendency) to, develop a retinal pathology.

Monitoring retinopathy may include monitoring and quantifying retinal gliosis, retinal degeneration or retinopathy related to neurodegenerative diseases including Parkinson's disease and Alzheimer's disease, primary retinopathies originating from the eye including retinoschisis, age-related macular degeneration and glaucoma, and secondary retinopathies originated from systemic diseases including diabetic retinopathy, hepatic retinopathy, renal retinopathy, hypertension, vascular diseases, congenital heart disease, autoimmune disorders including rheumatoid arthritis, multiple sclerosis, neurofibromatosis, Lyme neuroborreliosis, Down's syndrome, autism, sickle cell anaemia, infections with HIV and cytomegalovirus, thyroid disorders, or liver disorders.

“Disease status” refers to the extent of retinopathy or retinal degeneration in a particular transgenic non-human animal at a particular point in time. Disease status includes the stage of disease, including stage prior to onset, as well as the extent of disease. Disease status may be assessed by tracking fluorescence levels in the retinal glia of the transgenic animal over time and comparing and correlating changes of fluorescence levels with retinal pathology, including comparison with a transgenic animal that is free from a retinal pathology or a pre-disposition for a retinal pathology. As stated above, GFAP expression serves as a specific biomarker for retinal pathology, and thus fluorescence levels of the fluorescent protein expressed under control of the GFAP promoter may be used as an indicator of retinopathy.

The non-human transgenic animal may be any animal, including a mammal, including a rodent, including a mouse. In certain embodiments, the non-human animal is a mouse. The non-human animal may have a condition in which GFAP expression is elevated, due to reactive changes in the retinal glia. In certain embodiments, the animal is homozygous for the rd mutation, including mice having a FVB/N genetic background. The rd mutation results in retinal degeneration phenotype due to a mutation in the gene encoding the PDE6B enzyme subunit.

The non-human animal is a transgenic animal having a transgene comprising a GFAP promoter operably linked to a sequence encoding a fluorescent protein. The transgene is expressed in such a manner as to allow for detection of the fluorescent protein in the retinal glia cells or the in a live animal by examination of the retina. The glia cells of the retina are also referred to as the retinal glia and include Müller cells and astrocytes, and may also include non-myelinating Schwann cells of the optic nerve. Thus, the transgenic animal may have a nucleic acid molecule encoding a fluorescent protein under control of a GFAP promoter integrated into its genome.

In certain embodiments, the transgene comprises a GFAP promoter operably linked to a sequence encoding a fluorescent protein operably linked to a polyadenylation signal. It will be understood that the transgene construct will include the necessary regulatory elements to allow for the expression of the fluorescent protein within glial cells in the transgenic animal under control of the GFAP promoter.

A first nucleic acid sequence is operably linked with a second nucleic acid sequence when the sequences are placed in a functional relationship. For example, a coding sequence is operably linked to a promoter if the promoter activates the transcription of the coding sequence.

The GFAP promoter may be any promoter that directs expression of glial fibrillary acid protein. In particular embodiments, the GFAP promoter comprises the 2.2 kb 5′ region flanking the human GFAP gene, as described in Zhuo et al., 1997 and in Brenner et al., 1994.

The fluorescent protein may be any protein that fluoresces and that may be visualized when expressed in retinal glia cells by examining the retina of a live animal expressing such a protein. For example, the fluorescent protein may comprise GFP, GFP S65T, enhanced GFP (EGFP), EBFP, EBFP2, Azurite, mKalama1, ECFP, Cerulean, CyPet, YFP, Citrin, Venus, or Wet. The fluorescent protein in a particular embodiment is GFP, including humanized GFP, including the S65T mutant of GFP. A humanized protein refers to a protein in which the amino acid sequence is maintained but which is expressed from a coding sequence in which the codons have been optimized with respect to codon usage by human ribosomes.

The transgenic non-human animal further has a retinal pathology or a pre-disposition for a retinal pathology. Retinal pathology refers to disease or disorder of the retina which is related to retinopathy, as described above. Thus, the transgenic animal may have a genetic pre-disposition toward developing a primary or secondary retinopathy, or may have a primary or secondary retinopathy, or may have a retinal pathology including retinal or neural degeneration which may progress to a primary or secondary retinopathy.

Thus, the transgenic non-human animal may be generated by cross-breeding a transgenic animal expressing a fluorescent protein under control of the GFAP promoter with an animal that provides a genetic or molecular model for a primary or secondary retinopathy, including an animal that is used as a model for a neurodegenerative disease including Parkinson's disease and Alzheimer's disease, retinoschisis, glaucoma (including mouse model DBA/2J), diabetes, hepatitis, a renal disorder that may result in retinopathy, hypertension, a vascular disease, a cardiovascular disease, a pulmonary disorder, an autoimmune disorder including rheumatoid arthritis, multiple sclerosis, neurofibromatosis, or a thyroid disorder.

Alternatively, the transgenic animal may have a retinal pathology or retinopathy induced by exposure to a chemical, such as a neurotoxin. Several neurotoxins that induce retinal pathologies or retinopathy are known, including 1-methyl-4-phenyl-1,2,3,6-tetrahydropyrine (MPTP), kainic acid (KA) and 3,3-iminodipropionitrile (IDPN).

It has been established that glutamate receptor-related neurotoxicity can be induced by KA (Megumi Honjo, 2000a). KA has been used to model epilepsy as it has been shown to induce ongoing convulsions in rats as well as degeneration of the cornu ammonis (CA) neurons and hyperexcitability in the remaining CA neurons. When slowly administrated together with anticonvulsants, KA can be used to model Alzheimer's disease (AD) (Olney, 1990). One such study demonstrated visually apparent reductions in hippocampal pyramidal neurons on the intracerebroventricular administration of KA to neonatal rats (Dong et al., 2003). AD is a dementing disorder affecting the hippocampus and other limbic structures (Khachaturian, 1985), neocortical association areas, the visual cortex, and along the central visual pathway of the brain (Blanks J C, 1996b). Histopathological characteristics of AD typically comprise neuronal loss, neurofibrillary tangles, neuritic plaques and granulovacuolar degeneration (Khachaturian, 1985). Studies have demonstrated that AD patients suffer from visual impairment, collectively due to ganglion cell degeneration and optic neuropathy (Blanks et al., 1989) in the retina and central visual pathways (Blanks J C, 1996b). When injected into the vitreous of the eye, KA was shown to induce glutamate receptor-related neurotoxicity resulting in neuronal death, and up-regulation of GFAP in the retinal Müller cells (Megumi Honjo, 2000b).

Exposure to MPTP may result in degeneration of dopaminergic neurons in the substantia nigra pars compacta (SNpc) (Chen et al., 2003). Since the discovery of MPTP's ability to induce Parkinsonian symptoms in the 1980s, the neurotoxicant has been widely used to generate disease animal models for Parkinson's disease (PD). In particular, the mouse model for PD has been extensively utilized in the study of the mechanisms of dopaminergic neuron death and the development of experimental neuroprotective therapies (Przedborski et al., 2001; Przedborski and Vila, 2001; Bove et al., 2005). Currently, PD is regarded as the second most common degenerative disorder of the aging brain, with Alzheimer's disease being the most common. PD is characterized by symptoms of tremor at rest, slowness of voluntary movements, rigidity, and postural instability, and is attributed primarily to the loss of neurons of the nigrostriatal dopaminergic pathway, resulting in a deficit in brain dopaminergic level (Marin et al., 2005). PD is also associated with neurodegeneration of the visual system, as observed in patients with Parkinson's. Considerable psychophysical and electrophysiological evidence suggests that visual dysfunction in PD patients results from dopaminergic deficiency, possibly in the retina (Bodis-Wollner, 1990; Peters et al., 2000). Systematically administered MPTP has also been demonstrated to affect neuronal and glial cells in the retina. In one such study, microglia activation was observed in the inner outer retina within a week after systemic injection of MPTP in mice. The GFAP immunoreactivity of the glial cells also increased several days after injection (Chen et al., 2003).

IDPN is a neurotoxicant, which, upon administration to lab animals, induces symptoms of the human neurological disorder, Gilles de la Tourette syndrome. Gilles de la Tourette syndrome is characterized by fluctuating, involuntary motor and vocal tics. A prominent neurological syndrome of the neurotoxicant in animal models is the “ECC syndrome” (excitation, choreiform and circling movement) (Wakata et al., 2000). IDPN causes neurofilamentous axonopathy, which is characterized by the accumulation of neurofilaments in the proximal segments of neuronal axons and in the perikarya of myelinated cell bodies (Seoane et al., 1999). It has been shown to selectively induce necrosis in the olfactory mucosa following non-inhalation routes of exposure (Genter et al., 1996). Apart from progressive degeneration and reactive gliosis of the retina and colliculi, clouding of the cornea, vascular changes and detachment of the retina were also observed in the eyes of animal models (Seoane et al., 1999). With exception, reactive gliosis in astrocytes and Müller cells is also common feature associated with the IDPN neuropathy. This report describes a method for non-invasive imaging of the retinal gliotic response to the neurodegeneration examplified by the three neurotoxicants in the previously established GFAP-GFP transgenic mouse model (Zhuo et al., 1997) in the FVB/N background, which has a relatively high susceptibility for neurodegenerative diseases (Mineur Y S, 2002). Neurodegeneration is induced by injecting neurotoxicants which cause the same pattern of neurodamage and clinical symptoms as seen in diseases such as Alzheimer and Parkinson's (Landrigan P J, 2005; Bezard E, 2006; Novikova L, 2006). Unlike previous methods, this model would allow progressive and in vivo visualization of the astrocytes in the optic disc and central retina.

Similarly, retinal pathology or retinopathy may be induced by exposing the retina to radiation. For example, a laser may be used to induce retinopathy, including glaucoma, as described in Grozdanic et al., 2003.

Methods and techniques for constructing transgenes, transgene vectors and transgenic animals are known in the art, for example, as described in Sambrook et al. ((2001) Molecular Cloning: a Laboratory Manual, 3^{rd} ed., Cold Spring Harbour Laboratory Press). For example, transgenic mice may be produced by injection of a transgene construct into a pronucleus of a mouse zygote under conditions that allow for stable integration of the transgene into the mouse genome. Transgenic mice with having a GFAP promoter operably linked to the coding sequence for GFP and methods for making such mice have been described in U.S. Pat. No. 6,501,003 and in Zhuo et al., 1997.

The method involves detecting the fluorescence levels of the fluorescent protein in vivo, in the retina of the live transgenic animal, which animal is transgenic for a fluorescent protein expressed under the control of the GFAP promoter and which animal has a pre-disposition for or has a retinal pathology.

Detecting may be performed using known methods of detecting fluorescent markers in intact cells. For example, laser confocal microscopy methods may be used, as described in Zhuo et al., 1997 and in U.S. Pat. No. 6,501,003. Scanning laser opthalmoscopy methods may be used, for example employing a scanning laser opthalmoscope using a laser beam from a point source and detecting reflected light using a photomultiplier. Methods for imaging using a scanning laser opthalmoscope on rodents have previously been described (Hossain et al., 1998; Khoobehi and Peyman, 1999; Cordeiro et al., 2004; Genevois et al., 2004; Jaissle et al., 2001; Xu et al., 2003; Seeliger et al., 2005; Paques et al., 2006), and are set out in Example 1 below.

Detecting includes quantifying fluorescence levels of the fluorescent protein as well as qualitatively assessing expression of the fluorescent protein in the retinal glia of the transgenic animal. For example, fluorescent images detected using fluorescent microscopy techniques including opthalmoscopy methods may be captured by computer and quantified using standardly available imaging software. Methods that can be used to improve signal quantification under conditions where there is a high background fluorescence are described co-pending international application IMAGING AVERAGING, which claims priority to U.S. Provisional No. 60/924,162, filed May 2, 2007.

Detecting may be performed at intervals and over a period of time in a particular animal in order to monitor disease onset, disease progression, or disease regression. As mentioned above, fluorescence levels of the fluorescent protein detected at a later time point may be compared to fluorescence levels detected at an earlier time point. As well, fluorescence levels may be compared to fluorescence levels in a transgenic animal that expresses the fluorescent protein but does not have a retinal pathology or a pre-disposition for a retinal pathology. Such detecting may allow for establishment or definition of parameters, including cellular and molecular changes or events, associated with particular retinopathy related diseases or disorders or with particular disease stages, leading to methods that can be applied in a clinical setting relating to diagnosis or prognosis of a retinopathy related disease or disorder.

Optionally, the transgenic animal may be subjected to a potential treatment for a retinopathy related disease or disorder, and monitoring can be performed by detecting fluorescence levels of the fluorescent protein over a time course of potential treatment or before, during or after potential treatment.

The potential treatment may be any treatment that is to be tested for its effect on a retinopathy related disease or disorder, and may include dietary regimen, controlled environmental conditions, or administration of a potential therapeutic agent.

Thus, detecting may also be performed in the presence or absence of administration of a potential treatment including a potential therapeutic agent, including a drug, a pharmaceutical agent or a biologic agent, allowing for the monitoring of therapeutic effect and/or neurotoxicity of a potential treatment.

As well, therapeutic candidates for other diseases, disorders or conditions may be administered in the presently described methods and monitoring may allow for a determination of neurotoxicity of therapeutic agents for treatment of unrelated disorders.

A skilled person will be familiar with standard laboratory techniques and regimens for administering potential therapeutic candidates to the transgenic non-human animal used in these methods.

The described methods may be used in combination with other methodologies, including proteomic and metabolomic profiling of the transgenic animal, thus providing a molecular link between expression levels of the fluorescent protein (and thus expression levels of GFAP) and stages and types of retinopathy related diseases and disorders and potential treatments for such diseases and disorders.

Also presently contemplated is use of the transgenic non-human animal for monitoring retinopathy, in accordance with the above described methods.

The present methods and uses are further exemplified by way of the following non-limited examples.

### EXAMPLES

### Example 1

In this study, transgenic mice expressing green fluorescent protein (GFP) under the control of glial fibrillary acidic protein (GFAP) promoter were treated with three neurotoxicants, 1-methyl-4(2′-methylphenyl)-1,2,3,6-tetrahydropyridine (2′-CH₃-MPTP), kainic acid (KA) and 3,3-iminodipropionitrile (IDPN) to induce retinal gliosis. The progressive retinal gliosis was non-invasively imaged using a confocal scanning laser opthalmoscope (SLO) over a period of 2 weeks to visualize change in the GFP fluorescence in the glia of optic disc and central retina. The neurotoxicant-induced retinal gliosis was verified by the transgenic GFP and immunohistochemistry (IHC) staining of the endogenous GFAP on the retinal whole-mounts, and correlated with gliosis in other parts of the brain, including substantia nigra pars compacta (SNpc), striatum, hippocampus and olfactory bulb, which are the preferential targets for MPTP, KA and IDPN respectively. These findings indicate that the method described here using retinal gliosis in a transgenic mouse expressing green fluorescent protein (GFP) under the control of glial fibrillary acidic protein (GFAP) and real-time fluorescent imaging promoter is a useful pre-clinical tool for direct monitoring of the retinal gliosis, and for indirect prediction of gliosis occurring in the brain.

Materials and Methods

Transgenic GFAP-GFP mice: The generation and genotyping of the transgenic GFAP-GFP mice were done as previously described (Zhuo et al., 1997). Adult mice (8-10 weeks old) in the FVB/N background were used in the present study. Animal husbandry was provided by National University of Singapore animal holding unit. The experimental protocol covering the current study was approved by the Institutional Animal Care and Use Committee.

Neurotoxicants and dosing: In the current study, 1-methyl-4(2′-methylphenyl)-1,2,3,6-tetrahydropyridine (2′-CH₃-MPTP) which is a more potent analog of MPTP was used to induce gliosis in the adult brain (Abdel-Wahab, 2005). The test compounds 2′-CH₃-MPTP (M103), KA (K0250) and IDPN (317306) were purchased from Sigma-Aldrich (St. Louis, Mo., USA). Each mouse in the treatment group received either 4 intraperitoneal (ip) injections of 2′-CH₃-MPTP (15 mg/kg in saline, ip, once every 2 hours), a single injection of KA (25 mg/kg in saline, ip) or 3 injections of IDPN (500 mg/kg, ip, once daily). For each neurotoxicant-treated group (n=3), saline was used as a vehicle for the control group (n=3). Retinal imaging was subsequently performed over a period of 14 days.

Preparation of Animals: Mice were Anaesthetized by Ip Injections with 0.15 ml/10 g body weight of Avertin (1.5% 2,2,2-tribromoethanol; T48402) purchased from Sigma-Aldrich (St. Louis, Mo., USA), and their pupils dilated with a drop of 0.5% Cyclogyl® sterile ophthalmic solution (cyclopentolate hydrochloride, Alcon®, Puurs, Belgium). Custom-made PMMA hard contact lenses (from Cantor & Nissel (Northamptonshire, UK) were used to correct optical aberration and to avoid dehydration of the mouse eyes. Careful examination by an eye specialist before scanning laser opthalmoscope imaging ruled out the presence of any corneal or lens opacities.

Scanning laser opthalmoscope (SLO) imaging: For the work described here, the second version of Heidelberg Retina Angiograph (HRA II) scanning laser opthalmoscope (Heidelberg Engineering, Dossenheim, Germany) modified for use on mice was employed. The scanning laser opthalmoscope (SLO) is a fundus imaging technique based on the scanning of the fundus with a laser beam from a point source, while the reflected light is detected by a photomultiplier. Incident and reflected light follow a co-axial path. Therefore, more light can pass through small eyes than with conventional fundus cameras. Several reports of SLO imaging in rats (Hossain et al., 1998; Khoobehi and Peyman, 1999; Cordeiro et al., 2004; Genevois et al., 2004) and mice (Jaissle et al., 2001; Xu et al., 2003) have been published, with the latest study by (Seeliger et al., 2005) and (Paques et al., 2006) in evaluating mouse fundus with the first version of the HRA (or HRA I). The HRA II features the two Argon lasers in the short wavelength range (488 nm and 514 nm) and two infrared diode lasers in the long wavelength range (795 nm and 830 nm). The 488 and 795 nm lasers are used for fluorescein and indocyanine green angiography respectively. Appropriate barrier filters of 500 nm and 800 nm, respectively, remove the reflected light with unchanged wavelength and allow only the light emitted by the dye upon stimulation to pass through. The finest definition is 768×768 pixels at an optical resolution of 10 μm/pixel and coupled with three fields of view (nominal values of 15°, 20° and 30°). The focus is adjustable over a +12/−12 diopters range using step increments of 0.25 diopters. A video acquisition mode (48 ms to 96 ms per image) is available. Within any area of interest, a stack of tomographic images (z-scans) up to a maximum depth of 8 mm can be automatically acquired.

Retina wholemount and immunohistochemistry (IHC): After perfusing the mice with 1×PBS, followed by 4% fresh paraformaldehyde (in 1×PBS, pH 7.4), the brains were harvested and the eyes enucleated. The eyes were fixed immediately in 4% paraformaldehyde overnight at 4° C., after which they were dissected at the equator with the lens and vitreous removed. The retina was then dissected free from the choroid and sclera and whole mounted on a glass slide. Whole retina was mounted with Vectorshield mounting medium for fluorescence (Vector Laboratories, Inc., Burlingame, Calif., USA; H1000). The brains were first fixed in 4% paraformaldehyde for 4 hr at 4° C. and soaked in 30% sucrose at 4° C. overnight. The processed brain tissues were embedded in OTC freezing medium for sectioning on a cryostat (Leica Microsystems, Nussloch GmbH; CM-3050S). Coronal cryosections (10 μm) of the hippocampus (bregma −1.94 mm, interaural 1.86 mm), SNpc (bregma −3.16 mm, interaural 0.64 mm) and olfactory bulb (bregma 4.28 mm, interaural 8.08 mm) according to the Atlas of Mouse Brain (Franklin and Paxinos, 2001), together with the retina wholemounts were used for IHC using rabbit polyclonal antibodies (in 1:200 dilution) against GFAP (Dako, Z0334). Sections of the SNpc and striatum (bregma 0.62 mm, interaural 4.42 mm) were used for IHC using rabbit polyclonal antibodies (in 1:200 dilution) against tyrosine hydroxylase (TH; Chemicon, Calif., USA; Ab-152). The bound primary antibodies on the tissue sections were stained with a goat IgG conjugated to Texas-red (Abeam, Ab7088) at 1:100 dilution for 2 hr at room temperature, and visualized using confocal microscopy (LSM 510 META, Carl Zeiss Microimaging GmbH, Jena Germany).

Results

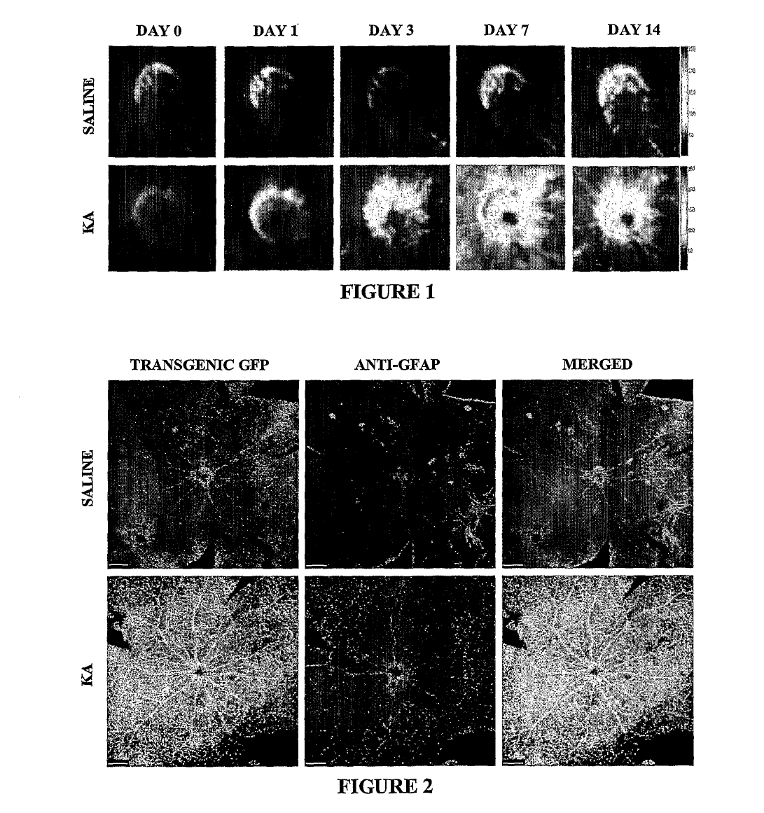

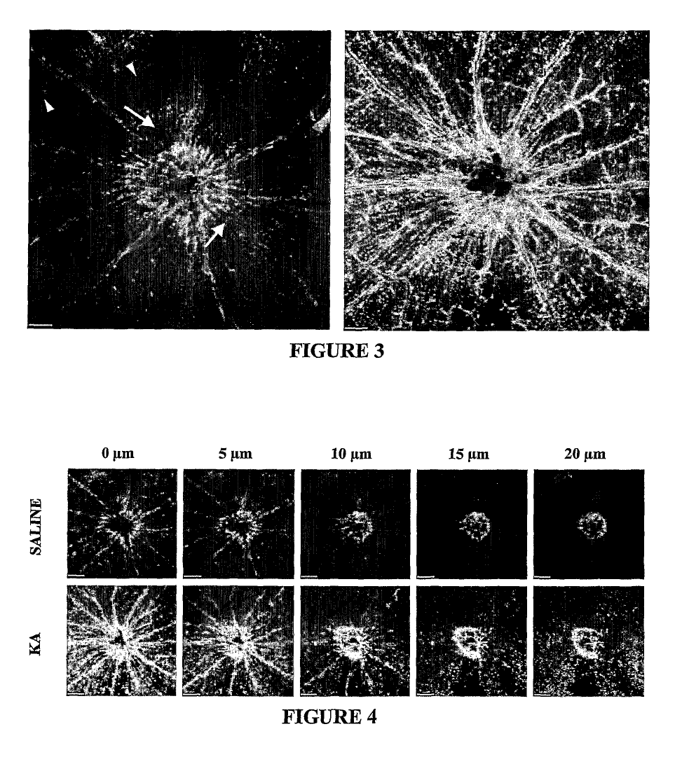

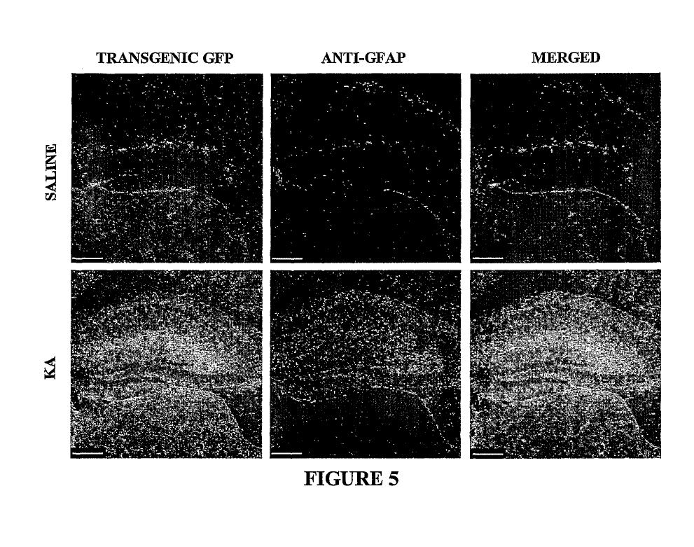

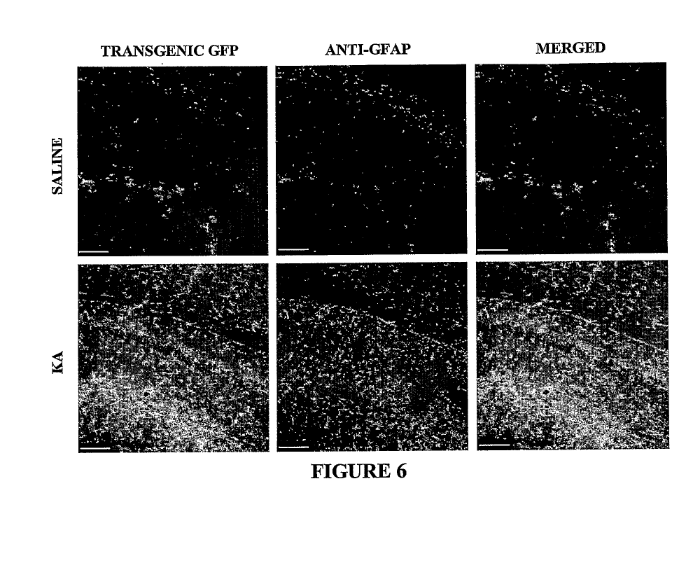

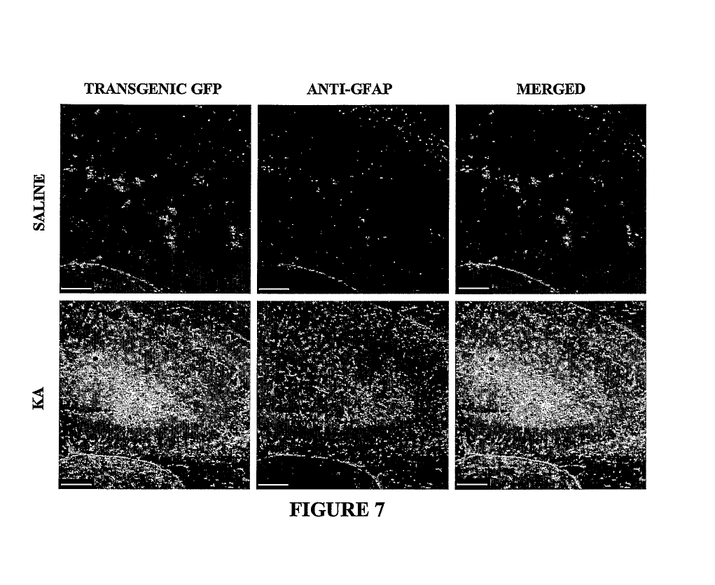

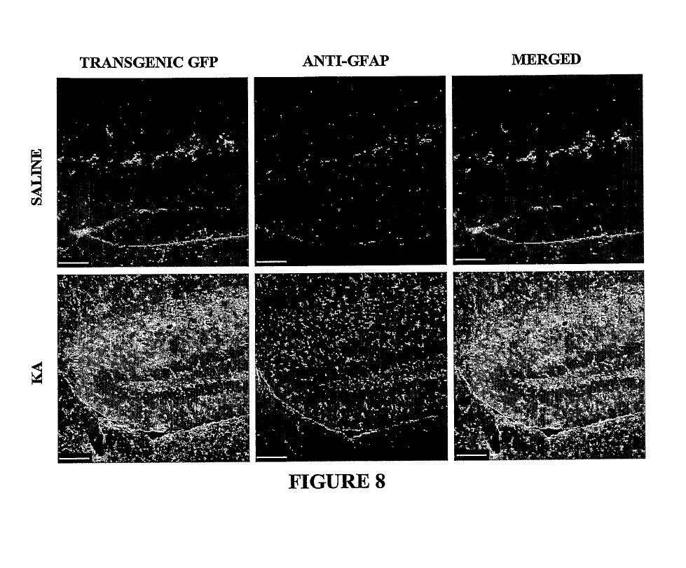

Molecular imaging of retinal gliosis induced by KA: Representative examples of retinal imaging in KA and saline-treated adult mouse are shown in FIG. 1. The optic disc of the neurotoxicant-treated mouse shows a gradual elevation of GFP fluorescence mainly at the rim of the optic nerve head, from day 1 after injection of the neurotoxicant until it reaches a maximum fluorescence at Day 7. Tiled images of retinal whole-mounts from KA-treated and saline-treated adult mice were analyzed. At 7 days following the administration of the neurotoxicant, severe gliosis with over-expressing transgenic GFP and endogenous GFAP in the nerve fiber layer (NFL) could be seen by the hypertrophy and hyperplasia state of the reactive astrocytic cells bodies and process in the optic disc and peripheral retina of the neurotoxicant-treated mouse (FIG. 2). As indicated by arrows in the merged transgenic GFP and anti-GFAP image of FIG. 3, the retinal blood vessels of the neurotoxicant-treated mouse enveloped by both the processes and cell bodies of the reactive astrocytes reveal web-like distribution and association between the astrocytes and blood vessels in the NFL, suggesting that the astrocytes are vascular glial sheaths and part of the blood-retina barrier. Interestingly, away from the optic nerve head, GFP signal is prominent in the cell bodies and processes whereas GFAP labeling is typically confined to just the processes of the stellate reactive astrocytes in the peripheral retina (denoted by arrowheads in FIG. 3). FIG. 4 shows a series of confocal images focused at regularly placed intervals of 5 μm through the depth of the whole-mount retinas from the KA-treated and saline-treated mice, demonstrating a qualitative difference in GFAP-GFP transgene expression between treated and control mice. Confocal images center around the optic disc illustrate the extent of gliosis in astrocytic cell bodies and processes from the NFL and ganglion cell layer at approximately 0-15 μm into the inner plexiform layer (15-20 μm) where numerous small foci of GFP fluorescence from the end feet (cell processes) of Müller cells can be seen. IHC staining of the frozen brain section by incubating with the GFAP antibody (red) and visualized together with the transgenic GFP reporter on the same focal plane demonstrates that KA induces severe reactive gliosis (i.e., up-regulation in GFAP and GFP). The acute gliosis when compared to the same areas of the saline control, displays extremely high level of GFP and GFAP expression in the entire hippocampus area (FIG. 5), especially in the localized region of CA1 (FIG. 6), CA3 (FIG. 7) and the dentate gyms (FIG. 8).

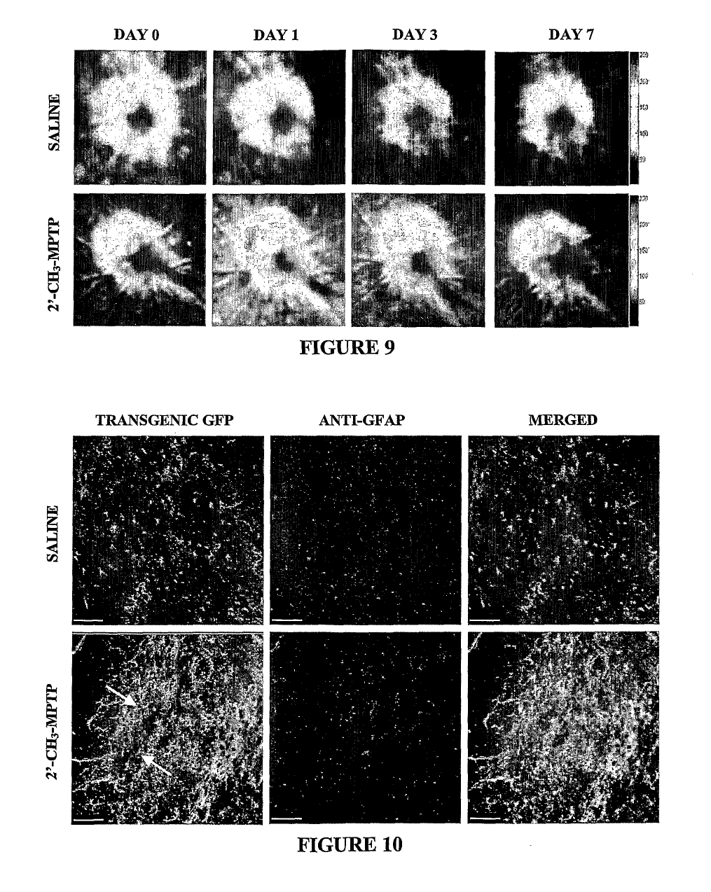

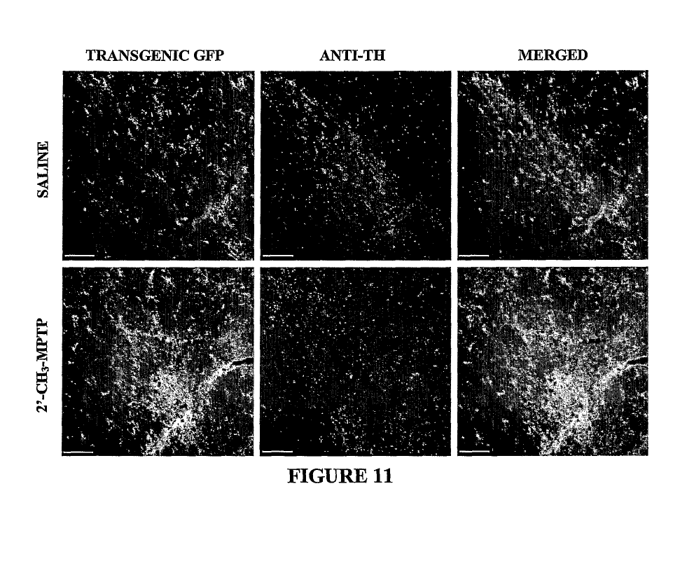

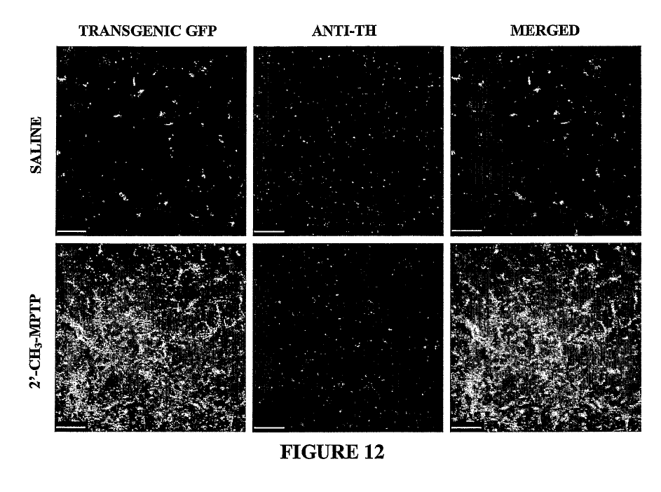

Molecular imaging of retinal gliosis induced by 2′-CH₃-MPTP: For the case of a second neurotoxicant 2′-CH₃-MPTP, fluorescent molecular retinal imaging of the optic disc clearly shows a maximal increase in GFP expression at 24 hours after administration of the neurotoxicant (FIG. 9). Regions of highest GFP fluorescence are mainly located in the fringe region of the optic disc where there is a congregation of axon fiber bundles and retinal vasculature. To identify the brain sub-region and cell type displaying gliosis, frozen sections encompassing the SNpc and striatum areas were prepared from the brains of the adult mice treated with 2′-CH₃-MPTP or saline, stained with either the GFAP antibody (red) or TH antibody (red) in conjunction with the transgenic GFP marker. 2′-CH₃-MPTP produces an increase in GFP and to a lesser extent, GFAP expression in the activated astrocytes at just a day after systemic administration of the neurotoxicant. As compared to the saline-treated mouse brain, gliosis occurs extensively in the substantia nigra (FIG. 10) of the neurotoxicant-treated mouse brain with a corresponding marked depletion of TH-immunoreactive midbrain dopaminergic neurons in the SNpc (FIG. 11). However, a substantial GFP increase in the striatum is accompanied by no detectable changes in level of TH expression (FIG. 12).

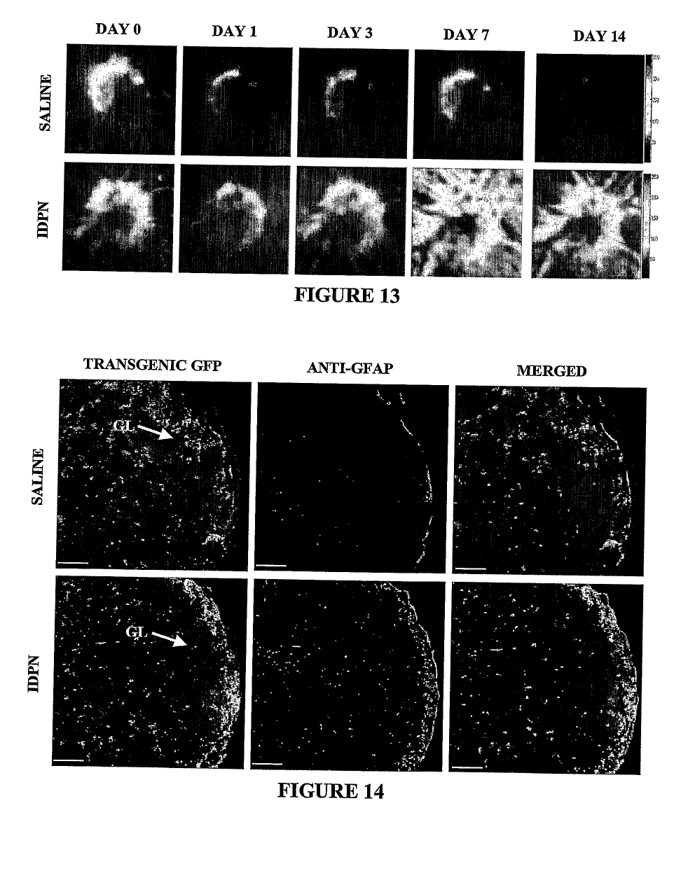

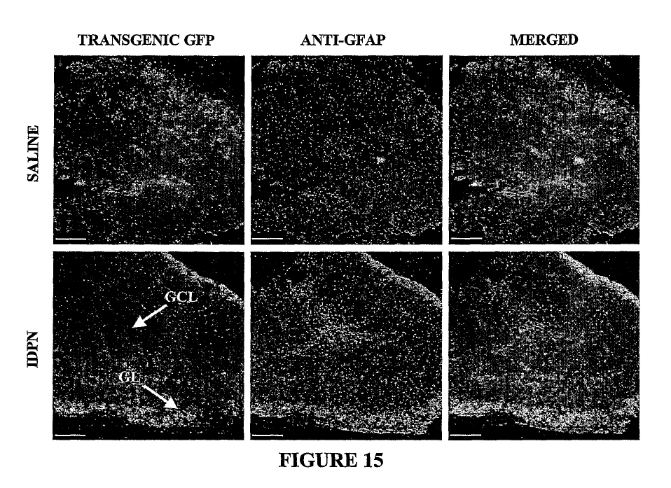

Molecular imaging of retinal gliosis induced by IDPN: In order to obtain additional support for the observations made with KA and 2′-CH₃-MPTP, neurotoxicant IDPN was used in a separate experiment. Similar to the KA case, the optic disc of the IDPN-treated mouse shows the largest elevation in GFP fluorescence at the Day 7 time point following the administration of the neurotoxicant, with the saline-treated mouse displaying only modest variation in fluorescence during the course of 2 weeks (FIG. 13). Tracking the onset of reactive gliosis in comparing corresponding sections of olfactory bulb in the saline-treated and IDPN-treated mouse, there are markedly more GFP-positive astrocytes co-localizing with GFAP-immunoreactive cells mainly in the glomerular layer (GL) of the olfactory bulb (FIG. 14). Besides congregating in the GL shown in FIG. 15, there is also a strong increase of GFAP-positive cells present in the granular cell layer (GCL).

Discussion

In mice, one can utilize the availability of in vivo retinal imaging tools to image the mouse intraocular vasculature (Ritter et al., 2005) and optic disc (Bruce E. Cohan, 2003) during development and disease. Knockout and transgenic mouse models are also being used to investigate retinal and neuronal degeneration (Jaissle et al., 2001; Helmlinger D, 2002). Transgenic mouse models under control of tissue-specific promoters over-expressing the GFP in different tissues can be incorporated into the study of specific disease. For example, crossing a GFP-expressed cone opsin promoter (Fei and Hughes, 2001) with a disease model can be used to study the loss of cones due to a degenerative disease over time. Similarly, vascular targets can be labeled with GFP. A transgenic producing GFP under control of smooth muscle α-actin promoter for example allows visualization of the retinal vessels without application of a dye (Tsai et al., 2002). Shown here in this study, the 2.2-kb human GFAP gene promoter, used as a gliosis pathological marker associated with a hGFP—S65T reporter gene, permits real-time fluorescent molecular imaging of both astrocytic and Miller cellular behaviors that enables monitoring and quantification of promoter activity over considerable periods of time. Using fluorescent molecular imaging of neurotoxicant-treated mouse eye, changes in the expression of the GFP in the mouse optic disc is observed in vivo, revealing important features about the gliosis process.

In both the retina and the brain, expression of the GFP transgene is regulated the same way as that of the endogenous GFAP gene. Molecular retinal imaging of the three neurotoxicants-treated mice has demonstrated to that the level of increased GFP expression in the astrocytes of the optic disc is significant enough to be readily visualized, monitored and quantified in vivo (FIG. 1). To substantiate the in vivo imaging data, transgenic GFP and endogenous GFAP levels of whole-mount retinas were examined by IHC. It was found that retinal gliosis observed in the whole-mount retina of KA-treated mouse is consistent with previously published studies where increased GFAP levels were detected in Müller cells and astrocytes after administration of KA (Sahel et al., 1991; Megumi Honjo, 2000b). Furthermore, acute gliosis in various regions of the brain like the SNpc, striatum and olfactory bulb, CA1, CA3 and dentate gyrus region of the hippocampus verified that the effect of the three neurotoxicants (KA, 2′-CH₃-MPTP, IDPN) elicits retinal gliosis, suggesting that the real-time fluorescent imaging method described here is a useful pre-clinical tool for direct monitoring of the retinal gliosis, and for indirect prediction of gliosis occurring in the brain.

As elucidated in the study, the present GFAP/GFP reporter system provides a method of in vivo visualization of retinal glia and their response to insults represents a real-time utility for pre-clinical screening of primary and secondary retinopathies, as well as evaluating the efficacy and neurotoxicity of drug compounds. It is feasible to obtain molecular retinal imaging of the peripheral retina and Müller cells at single cell resolution using a SLO mounted with a wide angle objective lens. Such an improved SLO configuration permits the analysis of change in retinal vascular structure and pattern in retinopathies of different origins. In particular, when complemented with electroretinography (ERG) and 3D-imaging modalities like Magnetic Resonance Imaging (MRI) and Positron Emission Tomography (PET), the current molecular retinal imaging methodology will not only be useful in detecting retinopathies, but also equally useful in obtaining molecular signatures for efficacy and neurotoxicity of drug compounds, as well as revealing more precise real-time information on the diseased areas deep within the brain. Furthermore, it may be possible to pinpoint a systemic disease with a retinopathy symptom to a specific organ by integrating data from both molecular retinal imaging and systemic (proteomic and metabolomic) profiling of serum biomarkers at a nano-scale (Hood et al., 2004).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

A R Irvine, L. L., D Schwartz, M Zarbin, F Ballesteros, and S Kroll, 1997. Retinal detachment in AIDS: long term results after repair with silicone oil. Br. J. Ophthalmol, 180-183.
Abdel-Wahab, M. H., 2005. Potential neuroprotective effect of t-butylhydroquinone against neurotoxicity-induced by 1-methyl-4-(2′-methylphenyl)-1,2,3,6-tetrahydropyridine (2′-methyl-MPTP) in mice. J. Biochem. Mol. Toxicol. 19, 32-41.
Antonetti D A, B. A., Bronson S K, Freeman W M, Gardner T W, Jefferson L S, Kester M, Kimball S R, Krady J K, Lanoue K F, Norbury C C, Quinn P G, Sandirasegarane L, Simpson I A., 2006. Diabetic retinopathy: seeing beyond glucose-induced microvascular disease. Diabetes September, 2401-2411.
Aristodemou P, S. M., 2006. Therapy insight: The recognition and treatment of retinal manifestations of systemic vasculitis. Nat Clin Pract Rheumatol. August, 443-451.
Bahceci U A, O. S., Pehlivanli Z, Yetkin I, Onol M., 2005. Changes in intraocular pressure and corneal and retinal nerve fiber layer thicknesses in hypothyroidism. Eur J Ophthalmol. September-October, 556-561.
Bezard E, G. I., Moratalla R, Gross C E, Jork R., 2006. 5-HT1A receptor agonist-mediated protection from MPTP toxicity in mouse and macaque models of Parkinson's disease. Neurobiol Dis July, 77-86.
Blanks, J. C., Hinton, D. R., Sadun, A. A., Miller, C. A., 1989. Retinal ganglion cell degeneration in Alzheimer's disease. Brain Research 501, 364-372.
Blanks J C, S. S., Torigoe Y, Porrello K V, Hinton D R, Blanks R H., 1996a. Retinal pathology in Alzheimer's disease. II. Regional neuron loss and glial changes in GCL. Neurobiol Aging May-June, 385-395.
Blanks J C, T. Y., Hinton D R, Blanks R H, 1996b. Retinal pathology in Alzheimer's disease. I. Ganglion cell loss in foveal/parafoveal retina. Neurobiol Aging May-June, 377-384.
Bodis-Wollner, I., 1990. Visual deficits related to dopamine deficiency in experimental animals and Parkinson's disease patients. Trends in Neurosciences 13, 296-302.
Bove, J., Prou, D., Perier, C., Przedborski, S., 2005. Toxin-induced models of Parkinson's disease. NeuroRX 2, 484-494.
Brenner M, K. W., Su Y, Besnard F, Messing A., 1994. GFAP promoter directs astrocyte-specific expression in transgenic mice. J Neurosci March; 14(3 Pt 1), 1030-1037.
Bruce E. Cohan, A. C. P., 1 Pentti T. Jokelainen, 2 and David F. Bohr3, 2003. Optic Disc Imaging in Conscious Rats and Mice. Investigative Ophthalmology and Visual Science, 160-163.
Burkhard, C., Gleichmann, M., Wilhelm, H., 2001. Optic nerve lesion following neuroborreliosis: a case report. European Journal of Ophthalmology 11, 203-206.
Chalam K V, S. V., 2004. Macular infarction a presentation of sickle cell crisis. Eye December, 1277-1278.
Chan C C, K. C., Kaiser-Kupfer M I, Parry D M, Gutmann D H, Zhuang Z, Vortmeyer A O., 2002. Loss of heterozygosity for the NF2 gene in retinal and optic nerve lesions of patients with neurofibromatosis 2. J Pathol. September, 14-20.
Chen, S. T., Hsu, J. R., Hsu, P. C., Chuang, J. I., 2003. The retina as a novel in vivo model for studying the role of molecules of the Bcl-2 family in relation to MPTP neurotoxicity. Neurochemical Research 28, 805-814.
Colakoglu Onder, T. B., Dayi Selcuk, Sozmen Bulent, Unsal Belkis, Maden Ahmet, Pasa Eser, Asian S. Leyla, 2005. Relationship between retinopathy and cirrhosis. World J Gastroenterol, 2193-2196.
Cordeiro, M. F., Guo, L., Luong, V., Harding, G., Wang, W., Jones, H. E., Moss, S. E., Sillito, A. M., Fitzke, F. W., 2004. Real-time imaging of single nerve cell apoptosis in retinal neurodegeneration. Proc Natl Acad Sci 101, 13352-13356.
Dong, H. X., Csernansky, C. A., Goico, B., Csernansky, J. G., 2003. Hippocampal neurogenesis follows kainic acid-induced apoptosis in neonatal rats. The Journal of Neuroscience 23, 1742-1749.
Dyer M A, C. C., 2000. Control of Müller glial cell proliferation and activation following retinal injury. Nature Neuroscience, 873-880.
Ek, U., Fernell, E., Jacobson, L., Gillberg, C., 1998. Relation between blindness due to retinopathy of prematurity and autistic spectrum disorders: a population-based study. Developmental Medicine and Child Neurology 40, 297-301.
Fei, Y. J., Hughes, T. E., 2001. Transgenic expression of the jellyfish green fluorescent protein in the cone photoreceptors of the mouse. Visual Neuroscience 18, 615-623.
Franklin, K. B. J., Paxinos, G. 2001. The Mouse Brain in Stereotaxic Coordinates. Academic Press, London.
Genevois, O., Paques, M., Simonutti, M., Sercombe, R., Seylaz, J., Gaudric, A., Brouland, J. P., Sahel, J., Vicaut, E., 2004. Microvascular remodeling after occlusion-recanalization of a branch retinal vein in rats. Investigative Ophthalmology and Visual Science 45, 594-600.
Genter, M. B., Owens, D. M., Carlone, H. B., Crofton, K. M., 1996. Characterization of olfactory deficits in the rat following administration of 2,6-dichlorobenzonitrile (dichlobenil), 3,3′-iminodipropionitrile, or methimazole. Fundamental and Applied Toxicology 29, 71-77.
Giordano N, D. E. M., Biasi G, Fioravanti A, Moretti L, Marcolongo R., 1990. Retinal vasculitis in rheumatoid arthritis: an angiographic study. Clin Exp Rheumatol March-April, 121-125.
Grozdanic S D, Betts D M, Sakaguchi D S, Allbaugh R A, Kwon Y H, Kardon R H., 2003. Laser-induced mouse model of chronic ocular hypertension. Invest Ophthalmol Vis Sci. 44(10), 4337-46.
Guidry, C., Medeiros, N. E., Curcio, C. A., 2002. Phenotypic variation of retinal pigment epithelium in age-related macular degeneration. Investigative Ophthalmology and Visual Science 43, 267-273.
Hammond S, W. J., Marcus D M, Prisant L M., 2006. Ophthalmoscopic findings in malignant hypertension. J Clin Hypertens (Greenwich) March, 221-223.
Hawes N L, S. R., Chang B, Davisson M, Heckenlively J R., John S W., 1999. Mouse fundus photography and angiography: a catalogue of normal and mutant phenotypes. Mol. Vis.
Heidrun Kuhrt, M. W., Andreas Reichenbach and Jan Albrecht, 2004. Rabbit retinal organ culture as an in-vitro model of hepatic retinopathy. Graefe's Archive for Clinical and Experimental Ophthalmology, 512-522.
Helmlinger D, Y. G., Picaud S, Merienne K, Sahel J, Mandel J L, Devys D., 2002. Progressive retinal degeneration and dysfunction in R6 Huntington's disease mice. Hum Mol Genet December 15, 3351-3359.
Hood, L., Heath, J. R., Phelps, M. E., Lin, B., 2004. Systems biology and new technologies enable predictive and preventative medicine. Science 306, 640-643.
Hossain, P., Liversidge, J., Cree, M. J., Manivannan, A., Vieira, P., Sharp, P. F., Brown, G. C., Forrester, J. V., 1998. In vivo cell tracking by scanning laser opthalmoscopy: quantification of leukocyte kinetics. Investigative Ophthalmology and Visual Science 39, 1879-1887.
Jaissle, G. B., May, C. A., Reinhard, J., Kohler, K., Fauser, S., Lütjen-Drecoll, E., Zrenner, E., Seeliger, M. W., 2001. Evaluation of the rhodopsin knockout mouse as a model of pure cone function. Investigative Ophthalmology and Visual Science 42, 506-513.
Karadimas P, H. E., Bouzas E A, 2003. Retinal vascular abnormalities in neurofibromatosis type 1. J Neuroophthalmol December, 274-275.
Kenison J B, F., Green W R., 1994. Retinal pathologic changes in Multiple Sclerosis. Retina, 445-451.
Khachaturian, Z. S., 1985. diagnosis of Alzheimer's disease. Archives of Neurology 42, 1097-1105.
Khoobehi, B., Peyman, G A., 1999. Fluorescent labeling of blood cells for evaluation of retinal and choroidal circulation. Ophthalmic Surgery and Lasers 30, 140-145.
Kirsch, L. S., Brownstein, S., de Wolff-Rouendaal, D., 1996. A histopathological, ultrastructural and immunohistochemical study of congenital hereditary retinoschisis. Canadian Journal of Ophthalmology 31, 301-310.
Kozak I, B. D., Cheng L, Kosobucki B R, Freeman W R., 2005. Objective analysis of retinal damage in HIV-positive patients in the HAART era using OCT. Am J. Ophthalmol. February, 295-301.
Landrigan P J, S. B., Butler R N, Trasande L, Callan R, Droller D, 2005. Early environmental origins of neurodegenerative disease in later life. Environ Health Perspect September, 1230-1233.
Lima C S, R. E. M., Silva N M, Sonatti M F, Costa F F, Saad S T, 2006. Risk factors for conjunctival and retinal vessel alterations in sickle cell disease. Acta Ophthalmol Scand. April, 234-241.
Liza-Sharmini A T, A. Z., Zilfalil B A., 2006. Ocular findings in Malaysian children with Down syndrome. Singapore Med J. January, 14-19.
Lucarelli M J, P. J., Arnold A C, Foos R Y., 1991. Immunopathologic features of retinal lesions in multiple sclerosis. Ophthalmology November, 1652-1656.
Lycke J, T. P., Frisen L., 2001. Asymptomatic visual loss in multiple sclerosis. J Neurol. December, 1079-1086.
Mader M M, C. D., 2006. Effects of induced systemic hypothyroidism upon the retina: regulation of thyroid hormone receptor alpha and photoreceptor production. Mol Vis August, 915-930.
Mansour, A. M., Bitar, F. F., Traboulsi, E. I., Kassak, K. M., Obeid, M. Y., Megarbane, A., Salti, H. I., 2005. Ocular pathology in congenital heart disease. Eye 19, 29-34.
Marin, C., Bove, J., Serrats, J., Cortes, R., Mengod, G., Tolosa, E., 2005. The kappa opioid agonist U50,488 potentiates 6-hydroxydopamine-induced neurotoxicity on dopaminergic neurons. Experimental Neurology 191, 41-52.
McCulley T J, L. B., Feuer W J., 2005. A comparison of risk factors for postoperative and spontaneous nonarteritic anterior ischemic optic neuropathy. J Neuroophthalmol. March, 22-24.
Megumi Honjo, H. T., Noriaki Kido, Masaru Inatani, Kazushiro Okazaki and Yoshihito Honda, 2000a. Expression of Ciliary Neurotrophic Factor Activated by Retinal Müller Cells in Eyes with NMDA- and Kainic Acid-Induced Neuronal Death. Investigative Ophthalmology and Visual Science, 552-560.
Megumi Honjo, H. T., Noriaki Kido, Masaru Inatani, Kazushiro Okazaki and Yoshihito Honda, 2000b. Expression of Ciliary Neurotrophic Factor Activated by Retinal Miller Cells in Eyes with NMDA- and Kainic Acid-Induced Neuronal Death. Investigative Ophthalmology and Visual Science, 552-560.
Milena Kuzmanovic, V. J. D., and Vijay P. Sarthy, 2003. GFAP Promoter Drives Müller Cell-Specific Expression in Transgenic Mice. Investigative Ophthalmology and Visual Science, 3606-3613.
Mineur Y S, C. W., 2002. Behavioral and neuroanatomical characterization of FVB/N inbred mice. Brain Res Bull January 1, 41-47.
Morgan J., H. R. W. R. O., 2005. Imaging techniques in retinal research. Exp Eye Res, 297-306.
Novikova L, G. B., Garris D R, Lau Y S., 2006. Early signs of neuronal apoptosis in the substantia nigra pars compacta of the progressive neurodegenerative mouse 1-methyl-4-phenyl-1,2,3,6-tetrahydropyridine/probenecid model of Parkinson's disease. Neuroscience June 19, 67-76.
Olney, J. W., 1990. Excitotoxic amino acids and neuropsychiatric disorders. Annual Review of Pharmacology and Toxicology 30, 47-71.
Paques, M., Simonutti, M., Roux, M. J., Picaud, S., Levavasseur, E., Bellman, C., Sahel, J. A., 2006. High resolution fundus imaging by confocal scanning laser opthalmoscopy in the mouse. Vision Research 46, 1336-1345.
Peters, S., Schweibold, G., Przuntek, H., Muller, T., 2000. Loss of visual acuity under dopamine substitution therapy. Neuro-ophthalmology 24, 273-277.
Przedborski, S., Jackson-Lewis, V., Naini, A., Jakowec, M., Petzinger, G., Miller, R., 2001. The parkinsonian toxin 1-methyl-4-phenyl-1,2,3,6-tetrahydropyridine (MPTP): a technical review of its utility and safety. Journal of Neurochemistry 76, 1265-1274.
Przedborski, S., Vila, M., 2001. MPTP: a review of its mechanisms of neurotoxicity. Clinical Neuroscience Research 1, 407-418.
Ritter, M. R., Aguilar, E., Banin, E., Scheppke, L., Uusitalo-Jarvinen, H., Friedlander, M., 2005. Three-dimensional in vivo imaging of the mouse ontraocular vasculature during development and disease. Investigative Ophthalmology and Visual Science, 3021-3026.
Roberts M R, S. M., Forrest D, Morreale de Escobar G, Reh T A., 2006. Making the gradient: thyroid hormone regulates cone opsin expression in the developing mouse retina. Proc Natl Acad Sci USA April 18, 6218-6223.
Ruggieri M, P. P., Polizzi A, Di Pietro M, Scuderi A, Gabriele A, Spalice A, Iannetti P., 2004. Ophthalmological manifestations in segmental neurofibromatosis type 1. Br J Ophthalmol November, 1429-1433.
Sahel, J. A., Albert, D. M., Lessell, S., Adler, H., McGee, T. L., Konrad-Rastegar, J., 1991. Mitogenic effects of excitatory amino acids in the adult rat retina. Experimental Eye Research 53, 657-664.
Sandstrom H, W. A., Eriksson M, Holmgren G, Lind L, Sandgren O., 1997. Angioid streaks are part of a familial syndrome of dyserythropoietic anaemia (CDA III). Br J Haematol. September, 845-849.
Satge D, S. D., Balmer A, Beck-Popovic M, Addor M C, Beckmann J S, Munier F L., 2005. Association Down syndrome-retinoblastoma: a new observation. Ophthalmic Genet September, 151-152.
Seeliger, M. W., Beck, S. C., Pereyra-Munoz, N., Dangel, S., Tsai, J. Y., Luhmann, F. O., 2005. In vivo confocal imaging of the retina in animal models using scanning laser opthalmoscopy. Vision Research 45, 3512-3519.
Seoane, A., Espejo, M., Pallas, M., Rodriguez-Farre, E., Ambrosio, S., Llorens, J., 1999. Degeneration and gliosis in rat retina and central nervous system following 3,3′-iminodipropionitrile exposure. Brain Research 833, 258-271.
Shakoor A, B. N., Shahidi M., 2005. Imaging retinal depression sign in sickle cell anemia using optical coherence tomography and the retinal thickness analyzer. Arch Ophthalmol. September, 1278-1279.
Takahashi H, G. T., Shoji T, Tanito M, Park M, Chihara E., 2006. Diabetes-associated retinal nerve fiber damage evaluated with scanning laser polarimetry. Am J Ophthalmol July, 88-94.
Tien Yin Wong, Anoop Shankar, Ronald Klein, Barbara E K Klein, Larry D Hubbard, 2004. Prospective cohort study of retinal vessel diameters and risk of hypertension. BMJ.
Topouzis F, C. A., Harris A, et al., 2006. Association of blood pressure status with the optic disk structure in non-glaucoma subjects: The Thessaloniki Eye Study. Am J Ophthalmol, 60-67.
Tsai, J. Y. Y., Yamamoto, T., Fariss, R. N., Hickman, F. I., Pagan-Mercado, G., 2002. Using SMAA-GFP mice to study pericyte coverage of retinal vessels. Investigative Ophthalmology and Visual Science 43, E-Abstract 1929.
V T Pham1, L. W., P McCluskey2, M C Madigan1 and P L Penfold1, 2005. Human retinal microglia express candidate receptors for HIV-1 infection. British Journal of Ophthalmology, 753-757.
Vogel J U, F. C., Wagner M, Gumbel H O, Theegarten D, Cinatl J Jr, Doerr H W., 2005. The human eye (retina): a site of persistent HCMV infection? Graefes Arch Clin Exp Ophthalmol. July, 671-676.
Vujosevic S, M. E., Pilotto E, Radin P P, Chiesa L, Cavarzeran F., 2006. Diabetic macular edema: correlation between microperimetry and optical coherence tomography findings. Invest Ophthalmol Vis Sci July, 3044-3051.
Wakata, N., Araki, Y., Sugimoto, H., Iguchi, H., Kinoshita, M., 2000. IDPN-induced monoamine and hydroxyl radical changes in the rat brain. Neurochemical Research 25, 401-404.
Wang L, C. G., Cull G, Dong J, Fortune B, 2002. Immunohistologic evidence for retinal glial cell changes in human glaucoma. Invest Ophthalmol Vis Sci April, 1088-1094.
Xu, H., Manivannan, A., Liversidge, J., Sharp, P. F., Forrester, J. V., Crane, I. J., 2003. Requirements for passage of T lymphocytes across non-inflamed retinal microvessels. Journal of Neuroimmunology 142, 47-57.
Yamakawa K, B. I., Lu Z, Watanabe Y, Amemiya T., 2001. Retinal vascular changes in rats with inherited hypercholesterolemia-corrosion cast demonstration. Curr Eye Res. 22, 258-265.
Zhang M, X. H., Atherton S S., 2005a. Murine cytomegalovirus (MCMV) spreads to and replicates in the retina after endotoxin-induced disruption of the blood-retinal barrier of immunosuppressed BALB/c mice. J Neurovirol. 11, 365-375.
Zhang M, X. H., Roon P, Atherton S S., 2005b. Infection of retinal neurons during murine cytomegalovirus retinitis. Invest Ophthalmol Vis Sci. 46, 2047-2055.
Zhuo, L., Sun, B., Zhang, C. L., Fine, A., Chiu, S. Y., Messing, A., 1997. Live astrocytes visualized by green fluorescent protein in transgenic mice. Developmental Biology 187, 36-42.

What is claimed is:

1. A method for monitoring retinopathy, comprising:
providing a live transgenic non-human animal having a retinal pathology or a pre-disposition for a retinal pathology, wherein a nucleic acid molecule encoding a fluorescent protein under control of a GFAP promoter is integrated into the genome of the transgenic non-human animal; and
detecting in vivo in the retinal glia of the transgenic non-human animal a first fluorescence level of the fluorescent protein at a first time point and a second fluorescence level of the fluorescent protein at a second time point.

2. The method according to claim 1, wherein the transgenic non-human animal is a mouse.

3. The method according to claim 1 or claim 2, wherein prior to detecting, the transgenic non-human animal is exposed to a potential therapy and wherein monitoring comprises monitoring for retinopathy regression, retinopathy recovery or a delay in retinopathy onset.

4. A method for monitoring retinopathy, comprising:
providing a live first transgenic non-human animal having a retinal pathology or a pre-disposition for a retinal pathology, wherein a nucleic acid molecule encoding a fluorescent protein under control of a GFAP promoter is integrated into the genome of the first transgenic non-human animal;
providing a live second transgenic non-human animal that is free from a retinal pathology or a pre-disposition for a retinal pathology, wherein a nucleic acid molecule encoding a fluorescent protein under control of a GFAP promoter is integrated into the genome of the second transgenic non-human animal; and
detecting in vivo in the retinal glia of the first transgenic non-human animal a first fluorescence level of the fluorescent protein and in the retinal glia of the second transgenic non-human animal a second fluorescence level of the fluorescent protein.

5. The method according to claim 4, wherein the first transgenic non-human animal and the second non-human transgenic animal is a mouse.

6. The method according to claim 4 or claim 5, wherein prior to detecting, the first transgenic non-human animal is exposed to a potential therapy and wherein monitoring comprises monitoring for retinopathy regression, retinopathy recover or a delay in retinopathy onset.

7. The method according to claim 6, wherein prior to detecting, the second non-human animal is exposed to the potential therapy.

8. The method according to claim 2 or claim 5, wherein the mouse has a FBV/N genetic background.

9. The method according to any one of claims 1 to 8, wherein the GFAP promoter is the 5′ 2.2 kbase region flanking the human GFAP gene.

10. The method according to any one of claims 1 to 9, wherein the fluorescent protein comprises GFP, GFP S65T, EGFP, EBFP, EBFP2, Azurite, mKalama1, ECFP, Cerulean, CyPet, YFP, Citrine, Venus, or Ypet.

11. The method according to claim 10, wherein the fluorescent protein comprises GFP S65T.

12. The method according to any one of claims 1 to 11, wherein the retinopathy comprises a primary retinopathy or a secondary retinopathy.

13. The method according to claim 12, wherein the primary retinopathy comprises retinopathy related to retinoschisis, age-related macular degeneration or glaucoma.

14. The method according to claim 12, wherein the secondary retinopathy comprises retinopathy related to Parkinson's disease, Alzheimer's disease, diabetic retinopathy, hepatic retinopathy, renal retinopathy, hypertension, a vascular disease, congenital heart disease, rheumatoid arthritis, multiple sclerosis, neurofibromatosis, Lyme neuroborreliosis, Down's syndrome, autism, sickle cell anaemia, HIV infection, cytomegalovirus infection, a thyroid disorder, or a liver disorder.

15. The method according to any one of claims 1 to 14, wherein the retinal pathology or pre-disposition for a retinal pathology is genetic.

16. The method according to any one of claims 1 to 14, wherein the retinal pathology is radiation-induced.

17. The method according to any one of claims 1 to 14, wherein the retinal pathology is chemical-induced.

18. The method according to claim 17, wherein the retinal pathology is induced by 1-methyl-4-phenyl-1,2,3,6-tetrahydropyrine, kainic acid or 3,3-iminodipropionitrile.

19. The method according to any one of claims 1 to 18, wherein detecting comprises scanning laser opthalmoscopy.

20. The method according to any one of claims 1 to 19, wherein detecting is performed at intervals and over a period of time in order to monitor retinopathy onset, retinopathy progression, retinopathy regression, retinopathy recovery or retinopathy prognosis.

21. The method according to any one of claims 1 to 20, wherein monitoring comprises monitoring therapeutic effect of a potential therapeutic agent.

22. The method according to any one of claims 1 to 20, wherein monitoring comprises monitoring neurotoxicity of a potential therapeutic agent.
